(19) United States
(12) Patent Application Publication
(10) Pub. No.: US 2019/0247055 A1
(43) Pub. Date: Aug. 15, 2019

(54) IMAGELESS ROBOTIZED DEVICE AND METHOD FOR SURGICAL TOOL GUIDANCE

(71) Applicant: ZIMMER GMBH, Winterthur (CH)

(72) Inventors: Bertin Nahum, Montpellier (FR); Eric Tassel, Montpellier (FR); Lucien Blondel, Montepellier (FR); Pierre Maillet, Carcassone (FR)

(21) Appl. No.: 16/280,661

(22) Filed: Feb. 20, 2019

Related U.S. Application Data
(63) Continuation of application No. 11/610,728, filed on Dec. 14, 2006, now abandoned.
Application No. 11/610,728 is a continuation of application No. PCT/EP05/52751, filed on Jun. 14, 2005.

(30) Foreign Application Priority Data
Jun. 15, 2004 (FR) 04/06491

(51) Int. Cl. A61B 17/15; A61B 34/00; A61B 34/20

(52) U.S. Cl. CPC A61B 17/154; A61B 34/70; A61B 2090/065; A61B 34/76; A61B 34/20

(57) ABSTRACT

An imageless robotized device for guiding surgical tools to improve the performance of surgical tasks is provided. The method of using the robotized device may include the steps of: collecting anatomical landmarks with a robot arm; combining landmarks data with geometric planning parameters to generate a position data; and automatically positioning a guiding tool mounted to the robot arm. Particular embodiments for a limb fixation device are also described.

### CROSS REFERENCE TO RELATED APPLICATIONS

This application is a continuation of U.S. application Ser. No. 11/610,728 filed on Dec. 14, 2006, which is a continuation of International Application Number PCT/EP2005/052751, filed on Jun. 14, 2005, which claims priority on French application number FR0406491, filed on Jun. 15, 2004, the disclosure of which is hereby expressly incorporated herein by reference.

### BACKGROUND

### 1. Field of the Invention

The present invention relates to the field of robotic-aided surgical systems and methods. More particularly, the present invention relates to mechanical guidance for an oscillating saw blade or a drill in a variety of surgical applications.

### 2. Description of the Related Art

Many surgical procedures in various specialties, such as orthopaedics, neurosurgery, and maxillofacial, for example, require precise bone cutting and/or drilling. For example, precise bone cutting and/or drilling may be required for knee surgeries, e.g., knee arthroplasty, tibial osteotomy, femoral osteotomy, or ligamentoplasty, for spine surgery, e.g., pedicular screw placement procedures, or for neurosurgery.

These procedures are traditionally carried out using motorized instruments, such as a surgical drill or an oscillating saw, for example, positioned and maintained either directly by the surgeon or using basic mechanical guides.

Total knee replacement (TKR) is an example of a surgical procedure that requires accurate cuts. In TKR, the surgeon resects the distal femur and the proximal tibia and implants prosthetic components to restore correct functionality of the knee joint.

Different approaches have been proposed to assist the surgeon during TKR. Navigation systems are based on a tracking system that locates the spatial position of trackers. Trackers are fixed on the femur, on the tibia, and on mechanical devices, such as cutting blocks and pointing tools, for example. The surgeon can visually follow the relative position of the tool with respect to the bones. In a first step, the surgeon registers anatomical landmarks and surfaces with a tracked pointer and defines the center of the hip joint by a kinematic procedure. The navigation system is then able to compute the mechanical axes of the bones and the optimal position for the different cuts. The surgeon fixes the cutting blocks on the bone with fixation pins and with the visual help provided by the navigation system.

Robotic systems have also been proposed to improve bone cutting during knee replacement surgery. One such robotic system utilizes a computer-assisted surgical system using a calibrated robot. The system uses a workstation which displays a 3D model of the patient's bones obtained from a CT scan of the leg and a modified industrial robot which directs the placement of prosthetic components. Positions of fiducial markers fixed on the bones are measured with a probe attached to the robot mounting flange. They serve to register the preoperative image data, e.g., a CT scan frame, with the position of the patient, e.g., a robot reference frame. After computing the optimal placement of the prosthetic component, the robot positions a drill guide where the holes for the cutting block are to be placed.

Another robotic device is disclosed in U.S. Pat. No. 5,403,319. This device includes a bone immobilization device, an industrial robot and a template attached to the robot mounting flange. The template has a functional interior surface corresponding to the exterior surface of the femoral component of a knee prosthesis. In the first step, the surgeon positions the template in the desired position of the prosthesis and the robot registers the position. In the second step, the system combines the registered position with a geometric database to generate coordinate data for each cutting task. The robot then positions a tool guide aligned for each specific task. The actual surgical task is carried out by the surgeon through the tool guide.

Other robotic systems have been proposed for performing total knee replacement, many of them using pre-operative image data of the patient. For example, ROBODOC™ and CASPAR™ surgical systems are active robots that automatically mill the bones, thereby autonomously realizing the surgical gesture. The Acrobot™ surgical system is a semi-active robot assisting the surgeon during the milling. The ROBODOC™, CASPAR™, and Acrobot™ systems are image based.

Other automated systems are proposed in combination with a navigation system. For example, the Praxiteles™ device from PRAXIM, the Galileo™ system from Precision Implants, and the GP System™ from Medacta International™ are all bone-mounted, require a large incision, and cannot work without a navigation system.

Other surgeries around the knee, such as a tibial osteotomy and ligament repairs, typically share the same issues as TKR: accurate cuts or drillings are required to restore knee functionality. In a tibial osteotomy, for example, a bone wedge is removed from the tibia to change the axis of the bone. The angular correction is determined pre-operatively on an X-ray. As for TKR, conventional instrumentation includes very basic mechanical guides.

### SUMMARY

The present disclosure provides an imageless system and method for surgical tool guidance by accurately positioning a guide mounted to a robot arm, such as a cutting guide, for example, used in knee replacement surgery for guiding an oscillating saw.

The method for surgical tool guidance may include the steps of collecting anatomical landmarks with a robot arm; combining landmarks data with geometric planning parameters to generate a position data; and automatically positioning a tool guide mounted to the robot arm.

In an exemplary embodiment, the device is a robotized surgical device used for the optimal positioning of a cutting or drilling guide.

The robotized device may be rigidly attached to the operating table by a specific fixation device.

In an exemplary embodiment, the robot arm presents at least six degrees of freedom and is adapted to receive a cutting and/or drilling guide and/or a pointing tool. The same instrument can be used both for pointing and guiding.

The robotized device positions the guide at the place where cutting or drilling may be carried out. Bone cutting or drilling is realized through the guide by a surgeon using an oscillating saw or a surgical drill.

In an exemplary embodiment, the robot arm includes a force sensor and can work in a cooperative mode in which the user has the ability to move the robot arm manually by grabbing it by its final part.

In another exemplary embodiment, movements of the guide in the cooperative mode can be restricted either to a plane for a cutting guide or to an axis for a drilling guide.

In another exemplary embodiment, the system may include a display monitor provided with a user communication interface to receive planning parameters from a user.

Anatomical landmarks data and planning parameters are combined to define the optimal position of the guide. For example, in TKR, the internal rotation of the femoral component is a planning parameter for implant positioning. The user communication interface could be, for example, a keyboard, a touch screen and/or a mouse.

In another embodiment, the device may also include an interface with a surgical navigation system being able to work from preoperative images of the bone, such as CT scan images or radiographic images, for example, or from intra-operative data. Data provided by the surgical navigation system are then used to generate position data for the guide. In this case, the use of a navigation system supplements the step of collecting anatomical landmarks with the robot. Data is provided from the navigation system through a communication interface in accordance to a predefined protocol.

In an exemplary embodiment, the guiding tool includes limited surfaces to reduce contact and friction with an oscillating saw while preserving an efficient guidance.

In another exemplary embodiment, the robotized device includes a limb fixation device adapted to ensure immobilization of the leg at two levels: at the level of the ankle with a toothed rack; and at the level of the knee with two pins screwed in the femoral or tibial epiphysis.

These means of fixation of the limb facilitate immobility of the leg during the steps of anatomical landmarks collection and bone cutting and/or drilling.

In one form thereof, the present disclosure provides an imageless device for guiding a surgical instrument relative to an anatomical structure, the device including an arm; a pointing tool releasably attachable to the arm, the pointing tool configured to provide data about the anatomical structure; and a control unit in communication with the arm, the control unit configured to receive information from the pointing tool and calculate a desired position for the arm.

In another form thereof, the present disclosure provides an imageless device for guiding a surgical instrument relative to an anatomical structure, the device including acquisition means for acquiring coordinates of a plurality of landmarks on the anatomical structure; processing means for processing the coordinates of the landmarks and generating a desired position for the surgical instrument relative to the anatomical structure based on the coordinates of the landmarks; and positioning means for positioning the surgical instrument in the desired position.

In yet another form thereof, the present disclosure provides a method for positioning a surgical instrument relative to an anatomical structure, the method including the steps of acquiring coordinates of a plurality of landmarks on the anatomical structure; calculating a desired position of the surgical instrument relative to the anatomical structure based on the acquired coordinates of the anatomical structure landmarks; and positioning the surgical instrument at the desired position relative to the anatomical structure based on the calculation step.

### BRIEF DESCRIPTION OF THE DRAWINGS

The above-mentioned and other features of this disclosure, and the manner of attaining them, will become more apparent and will be better understood by reference to the following description of embodiments of the disclosure taken in conjunction with the accompanying drawings, wherein:

FIG. 1 is a perspective view of the system of one embodiment of the present invention showing a mobile base, a robot arm with a force sensor and a tool mounted thereon, and a display monitor;

FIG. 2A is a perspective view of a pointing tool;

FIG. 2B is a perspective view of a guiding tool;

FIG. 2C is a perspective view of a pointing and guiding tool;

FIG. 3 is a perspective view of a fixation device for rigidly fixing the mobile base to the operating table;

FIG. 4A is a perspective view of a limb fixation device that rigidly holds the leg to the operating table;

FIG. 4B is a perspective view of the plate of the limb fixation device described in FIG. 4A;

FIG. 4C is a perspective view of the knee part of the limb fixation device described in FIG. 4A;

FIG. 4D is a perspective view of the ankle part of the limb fixation device described in FIG. 4A;

FIG. 5 is an exploded view of the pointing tool, the force sensor and the robot arm mounting flange;

FIG. 6 is a perspective view of the system of one embodiment of the present invention, further showing a patient positioned on an operating table; and

FIG. 7 is a block diagram showing various modules of the control software.

Corresponding reference characters indicate corresponding parts throughout the several views. The exemplifications set out herein illustrate embodiments of the disclosure and such exemplifications are not to be construed as limiting the scope of the invention in any manner.

### DETAILED DESCRIPTION

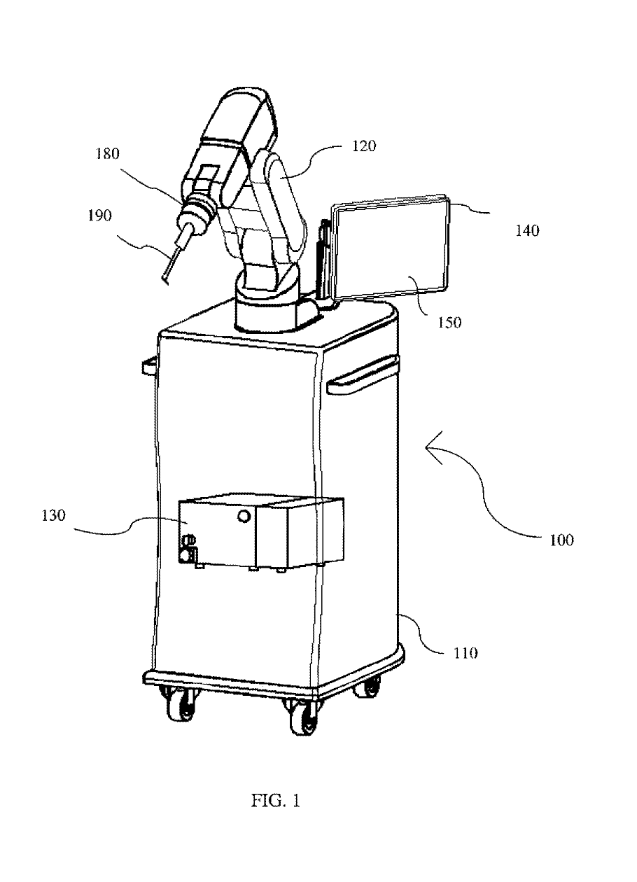

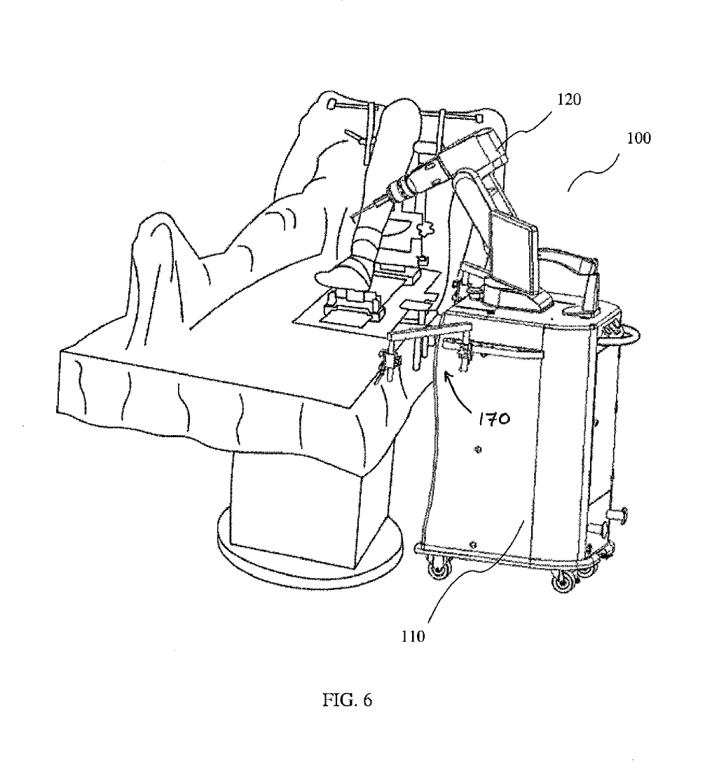

With reference to FIG. 1, it can be seen that an exemplary embodiment of the present invention generally includes robotized device 100 including mobile base 110; robot arm 120; control unit 130 inside mobile base 110 which controls robot arm 120 and allows a surgeon to manually input data through the use of interface 150, such as a touch screen, a mouse, a joystick, a keyboard or similar device, for example; display monitor 140; tool or instrument 190 and force sensor 180 mounted to a mounting flange of robot arm 120; and specific fixation device 170 (FIG. 6) to fix robotized device 100 to an operating table (FIG. 6).

Mobile base 110 ensures easy handling of robotized device 100 with its wheels and handles. In an exemplary embodiment, mobile base 110 includes immobilization pads or an equivalent device.

In an exemplary embodiment, robot arm 120 is a six joint arm. Each joint is provided with an encoder which measures its angular value. These data, combined with the known geometry of the six joints, allow computation of the position of the mounting flange of robot arm 120 and the position of the tool or instrument mounted to robot arm 120, which may be either a pointing tool, a guiding tool, or a pointing and guiding tool.

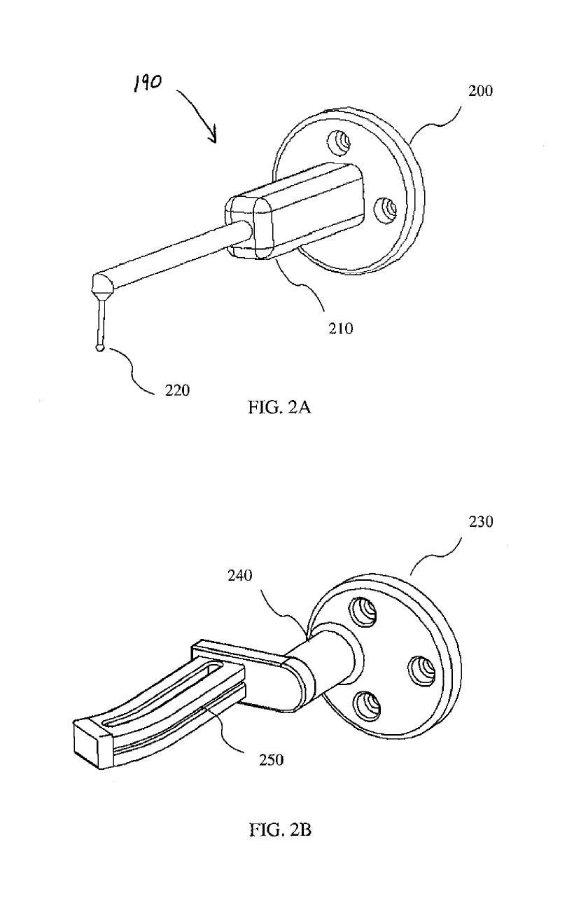

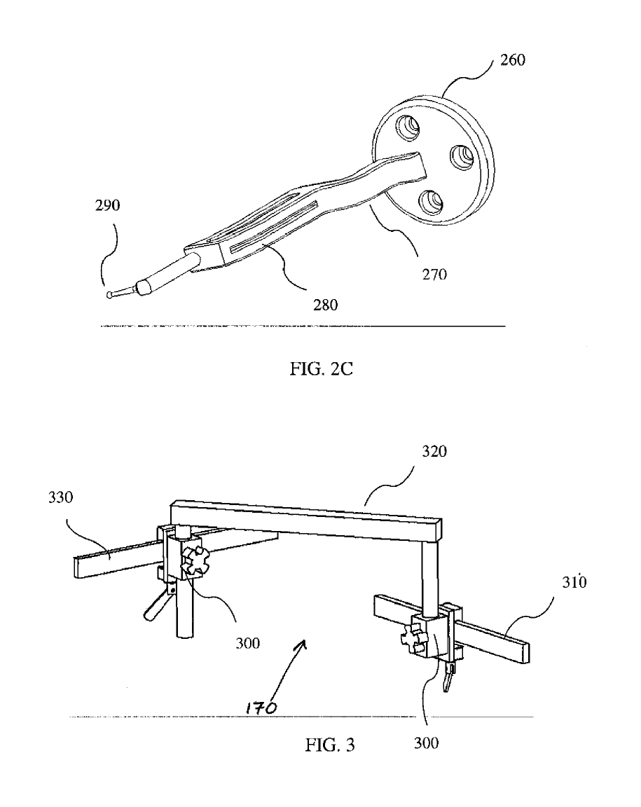

FIG. 2A illustrates pointing tool or instrument 190. The pointing tool 190 includes base plate 200; handle 210; and pointing sphere 220.

FIG. 2B illustrates a cutting guide or instrument. The cutting guide includes base plate 230; handle 240; and slit 250 to guide a saw blade.

FIG. 2C illustrates a pointing and guiding tool or instrument. The pointing and guiding tool includes base plate 260; handle 270; slit 280 to guide a saw blade; and pointing sphere 290.

The tools described in FIGS. 2A to 2C are three examples of pointing and/or guiding tools that may be utilized with the device shown in FIG. 1.

In an exemplary embodiment, robot arm 120 is rigidly attached to the operating table by specific base fixation device 170 (FIGS. 3 and 6). As shown in FIG. 3, base fixation device 170 may include two sets of clamps 300 adapted to operating table rail 310 and U-shape bars 320. Initially, the user installs one clamp 300 on operating table rail 310 and another clamp on mobile base rail 330. When clamps 300 are in place, the user inserts U-shape bar 320 in the cylindrical holes of clamps 300, locks clamps 300 in place, and locks U-shape bar 320 inside clamps 300 using the knobs.

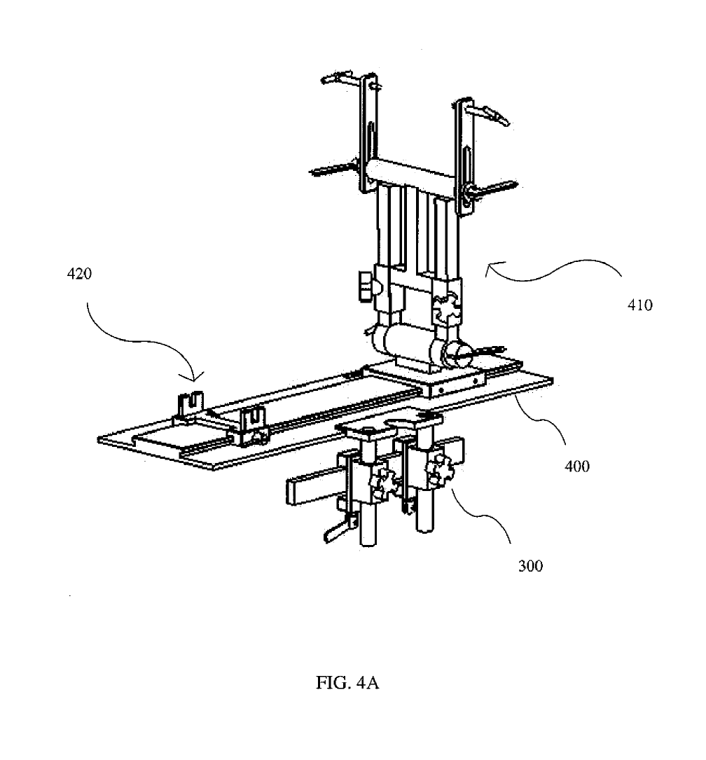

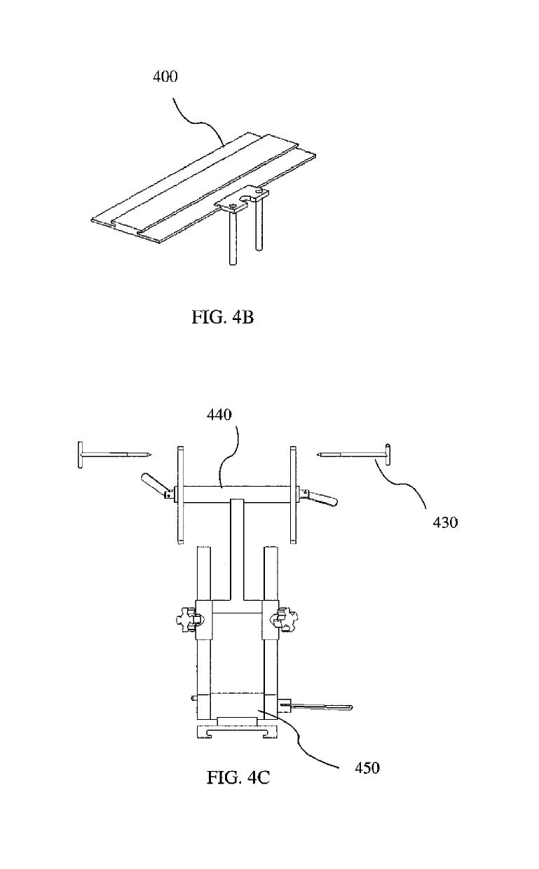

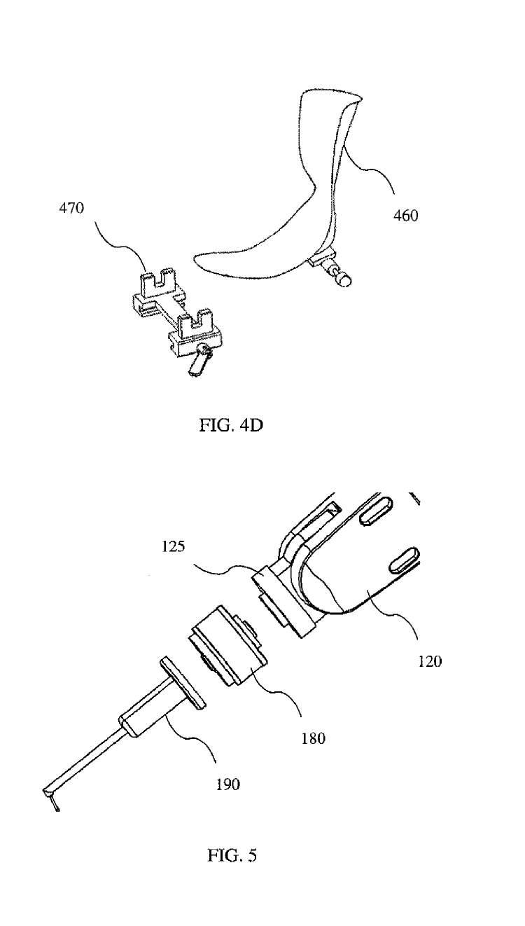

In an exemplary embodiment and referring to FIGS. 4A-4D, the system may include a limb fixation device to ensure immobility of the leg during the procedure. This limb fixation device allows an immobilization of the leg at two levels: at the level of the ankle with a toothed rack (FIG. 4D); and at the level of the knee with two pins screwed on the femoral or tibial epiphysis (FIG. 4C).

FIG. 4B shows main plate 400 of the limb fixation device. Main plate 400 is fixed on the operating table with two clamps 300. Knee fixation part 410 and ankle fixation part 420 can slide along the main plate 400 and be locked in place by screws.

FIG. 4C is a front view of the immobilizer for immobilizing the patient's leg at the level of the knee. The knee may rest on support bar 440. As bones are exposed in a knee replacement surgery, two pins 430 may be screwed either in the femoral epiphysis or in the tibial epiphysis. The position of support bar 440 can be adjusted vertically and locked with two knobs. The orientation can be adjusted from 0 to 90.degree. by rotating around main axis 450 and locked with one knob. The whole system can slide along plate 400.

FIG. 4D illustrates the immobilizer for immobilizing the patient's leg at the level of the ankle. The patient's foot and ankle are rigidly fixed with surgical tape or other sterile means to lock the foot in boot 460. Boot 460 is adapted to be clamped in carriage 470 that can slide along main plate 400 and be locked in place with a knob.

Both parts of the limb fixation device (ankle part and knee part) are independent but are used in combination to facilitate immobilization of the lower limb during the procedure.

In an exemplary embodiment, control unit 130 can set robot arm 120 in a cooperative mode in which a user is able to move robot arm 120 manually by grabbing it by its final part. With reference to FIG. 5, the system of the present invention may include force sensor 180 mounted to robot arm mounting flange 125. Force sensor 180 is adapted to receive a tool, such as pointing tool 190, for example. When the user grabs the tool and tries to move it in a direction, control unit 130 receives efforts measured by force sensor 180 and combines them with the position of robot arm 120 to generate the movement desired by the user.

Once robotized device 100 has been fixed to the operating table, the first step of the procedure is collecting anatomical landmarks on the patient. These anatomical landmarks are known by the surgeon. For example, in a TKR procedure, the malleoluses, the internal part of tibial tuberosity, the middle of the spines and the tibial plateaus are collected on the tibia; and the notch middle point, the distal and posterior condyles, and the anterior cortex are collected on the femur. FIG. 6 illustrates positions of the patient and of robotized device 100 at the beginning of the landmarks collection step for a TKR procedure.

During the landmarks collection step, control unit 130 sets robot arm 120 in cooperative mode and indicates through display monitor 140 the anatomical landmarks to acquire. The surgeon moves pointing tool 190 until contacting the required anatomical landmark. The surgeon validates the acquisition of the point coordinates using user interface 150. Control unit 130 then memorizes the coordinates of the point and its anatomical significance.

After the landmarks collection step, the surgeon inputs planning parameters through user interface 150. For example, in a TKR procedure, the surgeon chooses the model and the size of the prosthesis components and defines their positions and orientations relative to the mechanical axes of the femur and the tibia. Typical geometric parameters are varus/valgus angle, posterior slope and thickness of resection for the tibia and varus/valgus angle, flexion/extension angle, external rotation and thickness of resection for the femur.

In another embodiment, control unit 130 includes a data-processing interface that enables the system to be connected with another computer-assisted surgical system, like a navigation system. Navigation systems work with preoperative images of the bone, such as CT scan images, X-ray images, and fluoroscopy images, for example, or with intra-operative data. In the latter case, the system uses a 3D reconstruction algorithm based on the digitization of the bone. Data provided by the navigation system then replaces, or is combined with, the landmarks collection step data. Position of the guiding tool may be generated by the navigation system and transmitted to robotized device 100 in accordance with a predefined communication protocol.

Once the required position of the guide has been generated, the user mounts the guiding tool to robot arm 120. In an exemplary embodiment, a pointing and guiding tool is used such that the user does not need to change the tool between the landmarks collection step and the cutting or drilling step.

Robotized device 100 aligns the guide relative to the patient's anatomy, in accordance with the surgeon's planning. If the guiding tool is a cutting guide for a saw blade, robot arm 120 holds it in the chosen cutting plane. If the guiding tool is a drilling guide, robot arm 120 holds it along the chosen drilling axis.

In an exemplary embodiment, a planar cooperative mode can then be activated by the user to restrict movements of the guide in the plane. Similarly, an axial cooperative mode restricts movements of the guide along the axis. The user moves the guiding tool to an estimated optimal position, as control unit 130 restricts movements of robot arm 120 to a plane or an axis. Once this optimal position is reached, control unit 130 stops robot arm 120, thereby holding the guiding tool in place. Surgical tasks, such as bone cutting or drilling, for example, are carried out by the surgeon using a conventional instrument, such as an oscillating saw or a surgical drill, for example, through the guide.

In a TKR procedure, the same guiding tool may be used for the tibial cut and the five femoral cuts. In a tibial osteotomy procedure, the same guiding tool may be used for both tibial cuts.

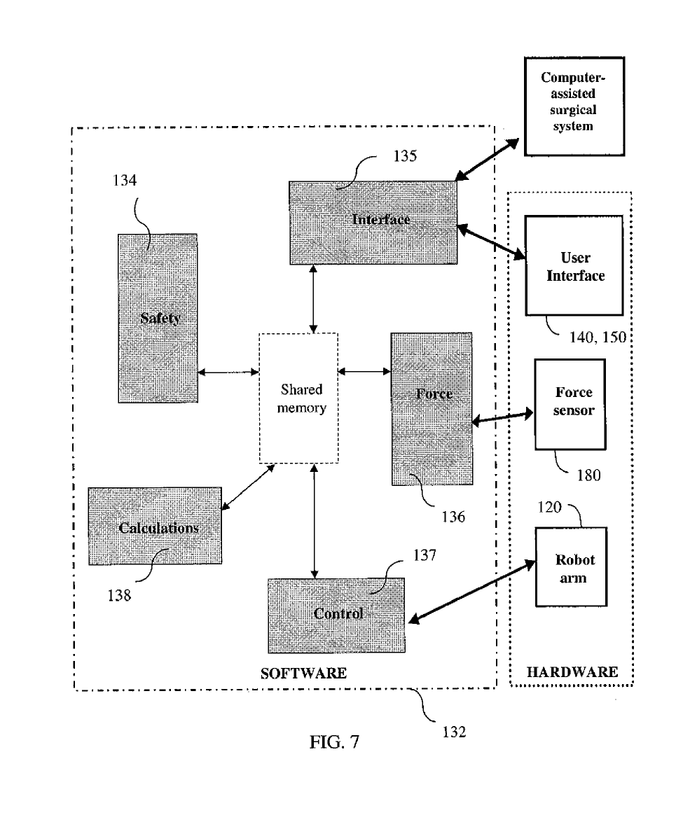

With reference to FIG. 7, control unit 130 runs control software 132 which exchanges data with elements of robotized device 100. Software 132 may communicate with the user through user interface 150 and display monitor 140. Software 132 may communicate with another computer-assisted surgical system, as described above, through a data-processing interface. Software 132 may communicate with force sensor 180 to regularly measure the efforts exerted by the user at the tool mounted to robot arm 120. Software 132 may communicate with robot arm 120 to control the position of robot arm 120.

Control software 132 may include five independent modules 134 to 138. In an exemplary embodiment, these modules run simultaneously under a real time environment and use a shared memory to ensure a good management of the various tasks of control software 132. Modules have different priorities, such as safety module 134 having the highest priority, for example.

Safety module 134 monitors the system status and stops robot arm 120 when a critical situation is detected, such as an emergency stop, software failure, or collision with an obstacle, for example.

Interface module 135 manages the communication between the surgeon and control software 132 through user interface 150 and display screen 140. Display screen 140 displays a graphical interface that guides the user through the different steps of the procedure. User interface 150 enables the user to have permanent control during the procedure, such as validating landmarks collection, defining planning parameters, and stopping robot arm 120 if needed, for example.

Force module 136 may monitor the forces and torques measured by force sensor 180. Force module 136 may be able to detect a collision with an obstacle and alert safety module 134.

Control module 137 manages the communication with robot arm 120. Control module 137 receives data encoder values of each joint and sends position commands.

Calculations module 138 does all the calculations necessary for the procedure. For example, in a TKR procedure, calculations module 138 reconstructs the mechanical axes of the bones combining anatomical landmarks data and statistical data. Calculations module 138 also defines the trajectory of robot arm 120 using direct and inverse kinematics.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

1. A robotized system for guiding a surgical instrument relative to an anatomical structure, the robotized system comprising:
a robot arm mounted on a base, the robot arm adapted to support a surgical instrument;
a limb fixation device; and
a control unit configured for tracking anatomical landmarks in a robot reference frame, the control unit in communication with said arm, said control unit calculating a desired position in said robot reference frame for said arm relative to a limb secured to the limb fixation device.

2. The robotized system according to claim 1, further including a fixation device adapted to fix the base to an operating table.

3. The robotized system according to claim 1, wherein the fixation device has at least one clamp adapted to be locked onto a rail of the operating table.

4. The robotized system according to claim 1, wherein the limb fixation device includes a knee fixation part adapted to a support a knee of a user.

5. The robotized system according to claim 4, wherein the knee fixation part is adapted to be connected to an operating table by a sliding joint.

6. The robotized system according to claim 6, wherein the sliding joint includes a rail.

7. The robotized system according to claim 6, wherein the sliding joint includes a clamp adapted to lock the sliding joint.

8. The robotized system according to claim 4, wherein the knee fixation part includes a knee rest support.

9. The robotized system according to claim 8, including a mechanism for vertical adjustment of the knee rest support.

10. The robotized system according to claim 8, wherein the knee fixation part includes pins adapted to contact the knee resting on the knee rest support.

11. The robotized system according to claim 8, including a mechanism for orientation adjustment of the knee rest support.

12. The robotized system according to claim 1, wherein the limb fixation device includes an ankle fixation part adapted to a support an ankle of a user.

13. The robotized system according to claim 12, wherein the ankle fixation part is adapted to be connected to an operating table by a sliding joint.

14. The robotized system according to claim 13, wherein the sliding joint includes a rail.

15. The robotized system according to claim 13, wherein the sliding joint includes a clamp adapted to lock the sliding joint.

16. The robotized system according to claim 12, wherein the ankle fixation part includes a boot.

17. The robotized system according to claim 12, wherein the limb fixation device includes a knee fixation part adapted to a support a knee of a user.

18. The robotized system according to claim 17, wherein the knee fixation part and the ankle fixation part are movable relative to one another by independent sliding joints.

19. The robotized system according to claim 18, wherein the independent sliding joints of the knee fixation part and the ankle fixation part are mounted on a common plate.

20. The robotized system according to claim 1, wherein said arm provides at least six degrees of freedom.
